(19) United States
(12) Patent Application Publication
(10) Pub. No.: US 2019/0290928 A1
(43) Pub. Date: Sep. 26, 2019

(54) APPARATUS AND METHOD FOR DETERMINING A DESIRED COIL POSITION FOR MAGNETIC STIMULATION

(71) Applicant: The Magstim Company Limited, Whitland (GB)

(72) Inventor: Matthew Biginton, Newcastle (GB)

(73) Assignee: The Magstim Company Limited, Whitland (GB)

(21) Appl. No.: 16/361,628

(22) Filed: Mar. 22, 2019

(30) Foreign Application Priority Data
Mar. 22, 2018 (GB) 1804557.5

(51) Int. Cl. A61N 2/00; A61N 2/02

(52) U.S. Cl. CPC A61N 2/006; A61N 2/02

(57) ABSTRACT

This application relates to a Magnetic Stimulation (MS) and preferably Transcranial Magnetic Stimulation (TMS) system. Examples according to this disclosure provide a simple, low cost and reliable system for appropriately positioning an MS coil arrangement into a treatment position or a position for diagnosis. An example system enables positioning of a MS coil arrangement relative to a wearer's body to a Desired Coil Position (DCP) and comprises a wearable apparatus comprising a support structure for positioning onto the body of a patient, the support structure carrying a plurality of sensors for measuring the proximity of a MS coil arrangement relative thereto to provide measured proximity values; a processor configured to compare DCP proximity values for each of the plurality of sensors representing the proximity of a MS coil arrangement to the sensors in the DCP to the measured proximity values and determine when the measured proximity values match the DCP proximity values thereby indicating a MS coil arrangement is in the DCP; an output arrangement for providing an indication to an operator when the MS coil arrangement is in the DCP.

### CROSS-REFERENCE TO RELATED APPLICATION

The present application claims the benefit of priority to Great Britain Patent Application No. 1804557.5, filed on Mar. 22, 2018, the entire contents of which are incorporated by reference herein for all purposes.

The present invention relates to Magnetic Stimulation (MS) and preferably Transcranial Magnetic Stimulation (TMS).

Magnetic stimulating of neuromuscular tissue is well known and comprises a stimulating coil made up of one or more windings, each having a plurality of turns which may generate a succession of electrical discharge pulses producing magnetic pulses which further induce electrical signals in the tissue. Such a coil arrangement is disclosed in U.S. Pat. No. 6,179,770 where the magnetic stimulator generally comprises a charging circuit, a capacitor, a discharge control and a winding which is of a size and power rating appropriate for the generation of magnetic fields sufficient to cause stimulation of a body portion. The individual winding or plurality of windings may be size adapted to fit partly over the cranium of a human patient in many applications, as well as being used for stimulation of other body parts. The coil arrangement including windings acts as an inductor and when connected to a stimulator which includes the capacitor provides an input voltage to the inductor which creates a circuit that passes an out of phase, sinusoidal voltage and current through the Transcranial Magnetic Stimulation (TMS) winding. An intense sinusoidal magnetic field is formed near the winding and is used to stimulate neurons in patients for medical and research applications. A typical coil arrangement comprises a single winding coil arrangement that may suitably be positioned on a patient's cranium. The winding is made up of a single wound conductive element connected to the capacitor via an elongate neck that allows positioning of the coil arrangement appropriate to the patient.

Such TMS coil arrangements are used for medical and research applications. A problem exists, particularly in TMS, of positioning the coil arrangement appropriately. There are many locations that are used to treat various medical conditions that involve TMS for example over the occipital nerve for the treatment of migraines and the dorsolateral prefrontal cortex (also known in the field as F3) for the treatment of major depressive disorder. Finding F3 can be achieved by using the so-called ‘Beam 3 method’ or the 5.5 cm method. In this example the 5.5 cm method is described.

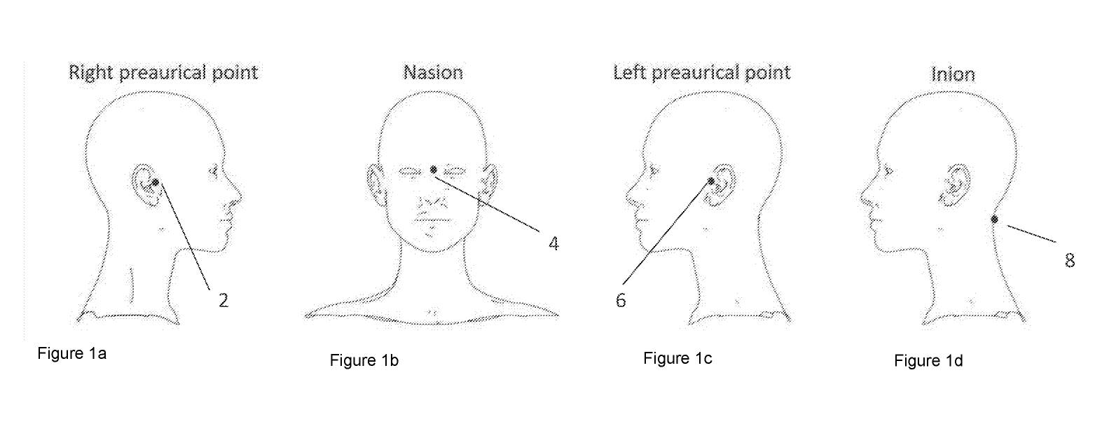

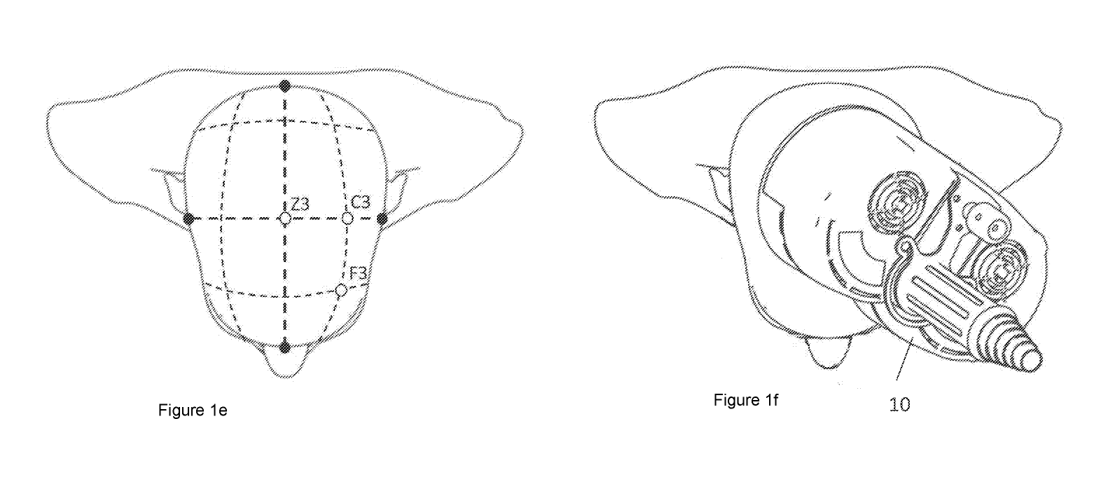

The intention is to initially position the TMS coil over the motor cortex area of the brain associated with the right thumb (C3), from which the F3 location can be found. FIGS. 1A-1D show the four bony landmarks on the head used to determine important landmarks to generate a head model based on these reference points. These are the right preaurical point 2, nasion 4, left preaurical point 6 and inion 8 respectively. FIG. 1E shows a position known as ‘Z3’ which is found by marking the mid distance from the nasion to inion adjusted by the mid distance from the right to left preauricular points. C3 is found by marking a point 20% of the distance from the right to left preauricular points. A procedure involving power adjustment and fine movements of the coil is followed until a thumb twitch is observed when the coil is pulsed over the patient specific area for the thumb found from the motor mapping starting from C3. This represents the minimum power setting to stimulate the cortex. This point is then typically manually marked by some means and then the treatment location is determined by moving the coil 5.5 cm forward from C3 to F3. Once this location is appropriately identified, an indicator is placed on the patient's cranium at F3 and the coil arrangement 10 directed to this location as presented in FIG. 1F.

However this is difficult to achieve as the coil arrangement obscures the view of the operator. TMS operators take a great deal of time and effort to ensure that the TMS coil is correctly placed in the correct location and to do this as in the manual method described above is costly in time and therefore there is a need for a better system. In addition getting the coil quickly onto the same treatment spot accurately on subsequent patient visits is the key to an efficient clinical practice.

Navigation systems have therefore been developed to achieve easier coil arrangement positioning. A relatively simple method is to provide a patient with a cap which is worn on the head. At the first treatment the appropriate position for treatment is located and marked on the cap in the known way of finding the motor threshold and treating at a predetermined location from this. On subsequent treatment sessions the cap is worn and treatment carried out at the same location as marked. However, such a technique is susceptible to the cap being incorrectly positioned on the head. In addition, as the TMS coil arrangement is large the operator cannot see the underside of the coil arrangement and it is difficult to ensure that the point between the two windings in the TMS coil arrangement is actually positioned in alignment with the treatment location. Accordingly, treatment may be carried out at the wrong location thereby reducing efficacy.

Other techniques include infrared tracking cameras that track reflective markers positioned on the coil arrangement and on the patient's head. However in use reflective markers may lose their reflective coating potentially causing error in measurements. Furthermore, interference is common through changes in light conditions such as sunlight entering a room, and in addition the required cameras are high cost.

A simple, low cost and reliable system is therefore needed for appropriately positioning a MS coil arrangement, and preferably a TMS coil arrangement, preferably into a treatment position or optionally a diagnostic position.

According to the present invention there is a system for enabling positioning of a Magnetic Stimulation (MS) coil arrangement relative to a wearer's body to a Desired Coil Position (DCP) comprising:
a wearable apparatus comprising a support structure for positioning onto the body of a patient, the support structure carrying a plurality of sensors for measuring the proximity of a MS coil arrangement relative thereto to provide measured proximity values;
a processor configured to compare DCP proximity values for each of the plurality of sensors representing the proximity of a MS coil arrangement to the sensors in the DCP to the measured proximity values and determine when the measured proximity values match the DCP proximity values thereby indicating a MS coil arrangement is in the DCP;
an output arrangement for providing an indication to an operator when the MS coil arrangement is in the DCP.

Accordingly, in operation when the current position of the MS coil arrangement matches the DCP administering of magnetic stimulation may be performed. A match may be a match within predetermined error ranges, and/or may be different error ranges for each sensor.

The DCP is preferably a treatment position, however may be a diagnostic position.

The output arrangement preferably guides a MS coil arrangement to the DCP. Audible and/or visual indicators may be provided. The MS coil arrangement is beneficially a Transcranial Magnetic Stimulation (TMS) coil arrangement. The DCP proximity values are beneficially predetermined.

The wearable apparatus is preferably arranged to seat onto the body of a wearer, and preferably the head of a wearer.

The output arrangement preferably comprises a graphical display and presents on the graphical display a graphical representation of a MS coil arrangement in the DCP and a graphical representation of the current MS coil arrangement position. The operator can therefore be guided in locating the MS coil arrangement into the DCP. An additional audible or visual indication may be provided when the DCP is matched by the current position of the MS coil arrangement. The operator may then lock the MS coil arrangement in the DCP and perform the treatment. The provision of a live graphical representation of the MS coil arrangement as it moves through the area around a patient's head and toward the DCP is beneficial as this enables an operator to also be guided as to both position and orientation of the MS coil arrangement. The system is preferably further arranged to present a graphical representation of the wearer's head on the graphical display.

It will be appreciated that the measured values may be within predetermined tolerance values compared to the DCP reference values.

Also according to the present invention there is a wearable apparatus for use in positioning of a Magnetic Stimulation (MS) coil arrangement relative to a wearer's body, the wearable apparatus comprising a support structure for positioning onto the body of a wearer, the support structure carrying two or more sensors for measuring the proximity of a MS coil arrangement.

The sensors are carried by the support structure such that the proximity of a coil arrangement can be determined in a measurement zone around a wearer's head. The DCP of a wearer is typically referred to as the F3 point over the dorsolateral prefrontal cortex. The wearer may be termed a patient.

The support structure is preferably arranged to support the two or more sensors at the periphery of a measurement zone, and the two or more sensors are supported such that they are inwardly directed into the measurement zone.

The support arrangement is preferably arranged to support the sensors such that a first and a second sensor emit an output in a first and a second direction respectively, and wherein the first and second direction are converging. The first and second sensors are therefore beneficially angled relative to one such that the output emitted is towards a converging point. This means that an output from each sensor can be utilised to more accurately determine the live/current position and orientation of the MS coil arrangement.

The support structure is preferably arranged to seat onto a wearer's nose and preferably one or both of a wearer's ears. There is a significant benefit associated with such a configuration of the support structure where in seating on a wearer's nose repeated positioning of the wearable device can be achieved for each treatment session. As a result of being able to ensure accuracy of positioning of the support structure and thus the sensors, the repeated positioning of a MS coil arrangement in the desired treatment position can be achieved. It is preferable that the support structure is supported by both the wearer's nose and both ears, thereby providing stability of the wearable device in use.

The support structure preferably comprises a first seating portion for seating onto a wearer's nose, and further comprises at least one arm extending to a distal end having a second seating portion for seating onto a wearer's ear. The arm may be adjustable in length. This has a first advantage in that the wearable device is comfortable for a wearer, and secondly ensures that a marker, if utilised, may be consistently located adjacent the left/right preaurical point of a wearer meaning determining of an initial treatment position can be better determined.

The support structure is preferably arranged to carry the plurality of sensors such that the sensors emit an output into the measurement zone around a patient's head. The support structure is preferably arranged such that when positioned on the body the two or more sensors are spaced apart from the body. The output may reflect from the MS coil arrangement and thus proximity determined. The support structure preferably comprises one or more sensor carrying portions arranged to extend away from a wearer's head such that the proximity sensors are spaced apart from a patient's head. The or each sensor carrying portion preferably extends away from the or each arm. The sensors are preferably spaced apart from the one or more arms. The sensor carrying portion preferably extends away from the head when the support structure is mounted to a wearer's head.

One or each of the two or more sensors may be provided as one or more sensor arrays. This is beneficial as increasing the number of individual proximity sensors each providing an independently measured proximity of a coil arrangement increases the accuracy of positioning of a MS coil arrangement into the DCP.

The wearable device comprises one or more markers that provide reference points for a wearer's head. This is beneficial as assists in location of the DCP, and further optionally enables increased accuracy of presentation of a graphical representation of the wearer's head on a graphical display.

The wearable device preferably comprises multiple markers. A marker is preferably provided on the or each of the arms of the wearable device.

The sensors preferably comprise proximity sensors.

The proximity sensors preferably comprise time of flight sensors.

Also according to the present invention there is a method of positioning a MS coil arrangement relative to a wearer's body to a Desired Coil Position (DCP) comprising:
positioning a wearable apparatus comprising a support structure onto the body of a patient, the support structure carrying a plurality of sensors for measuring the proximity of a MS coil arrangement relative thereto to provide measured proximity values;
comparing the measured proximity values to each of DCP reference values representing the proximity of a MS coil arrangement to the sensors in the DCP;
outputting an indication to an operator when the measured proximity values match the DCP proximity values thereby indicating a MS coil arrangement is in the DCP.

It will be appreciated that a match between measured and DCP proximity values may be a match within predetermined error ranges.

Aspects of the present invention will now be described by way of example only with reference to the accompanying drawings in which:

FIGS. 1A-F are representations of reference points used for determination of a treatment position represented in FIG. 1E, and FIG. 1F includes a TMS coil arrangement in the DCP.

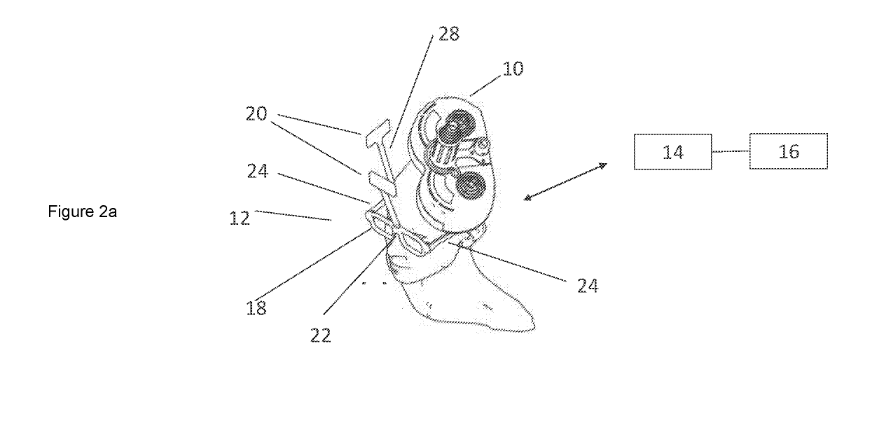

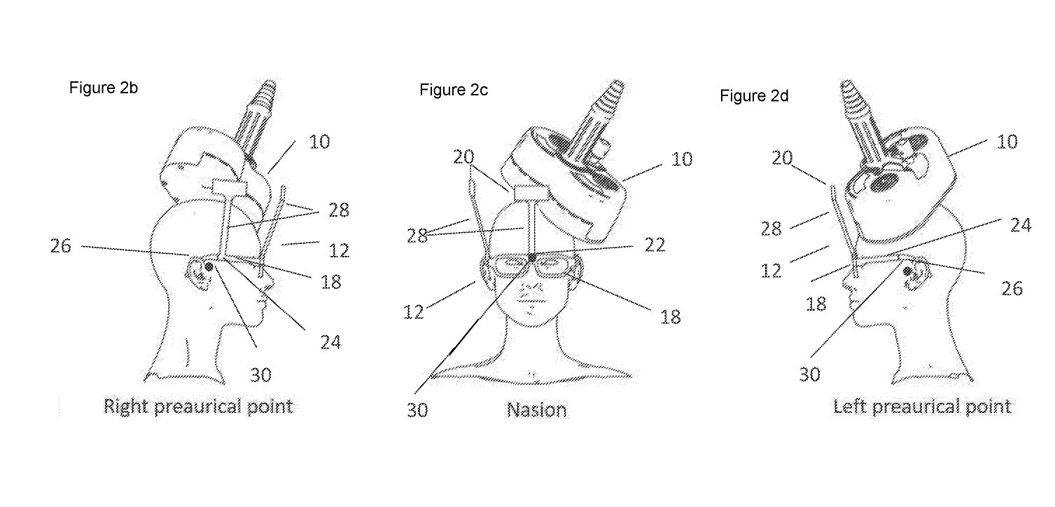

FIGS. 2A-D are schematic representations of an illustrative embodiment of the present invention.

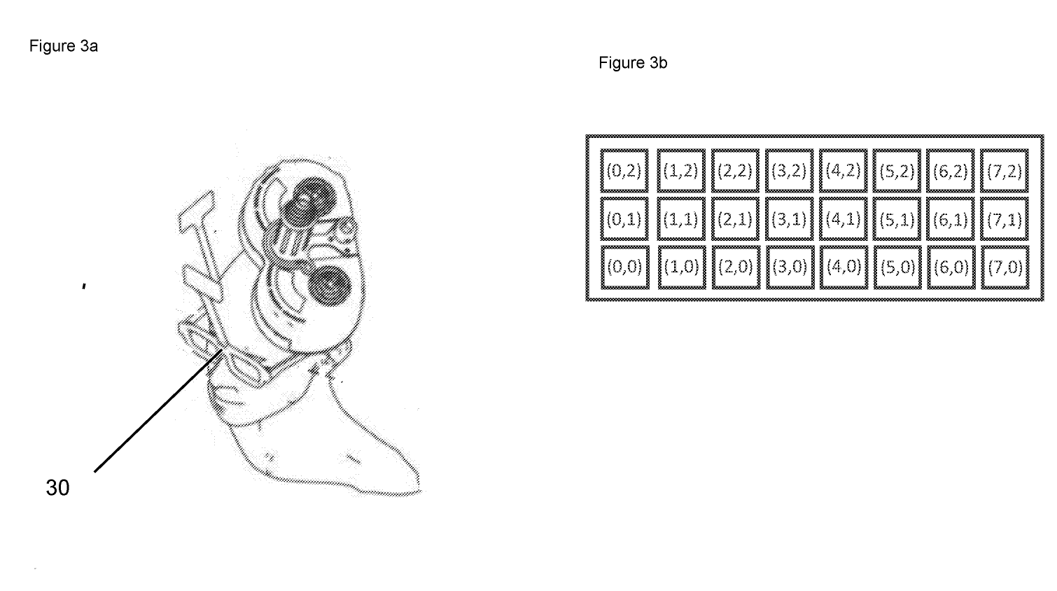

FIG. 3A is a schematic representations of a wearable apparatus according to an illustrative embodiment and FIG. 3B is an illustration of a proximity sensor array.

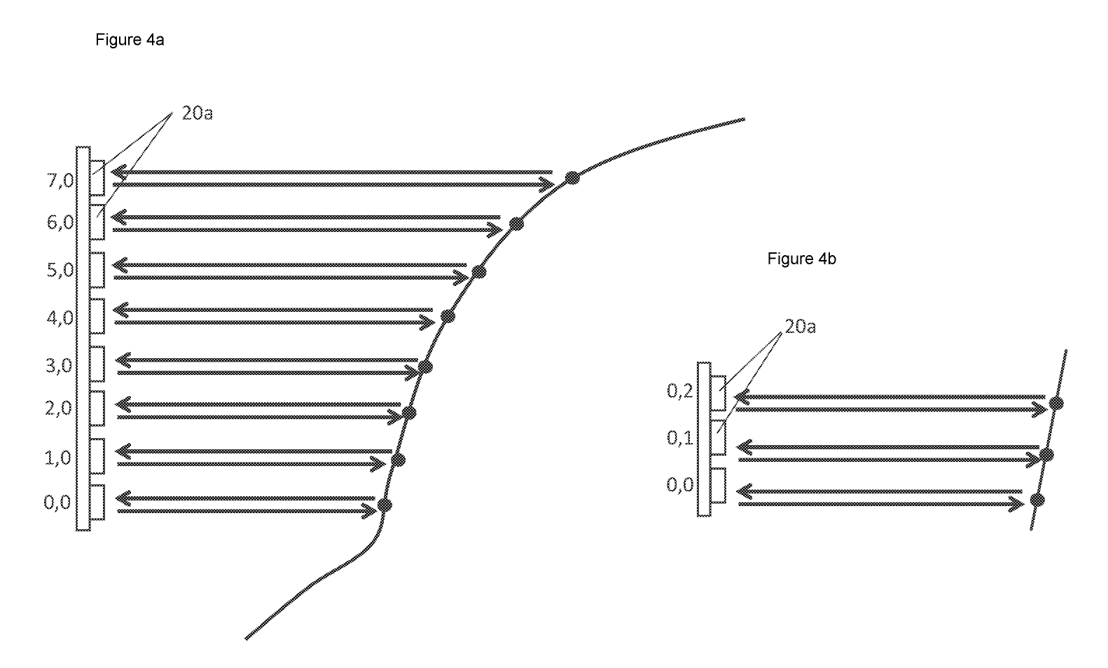

FIGS. 4A and 4B are schematic representations of the path taken of the light from individual sensors in a sensor array from a plan and side view respectively for an array with twenty four individual sensors.

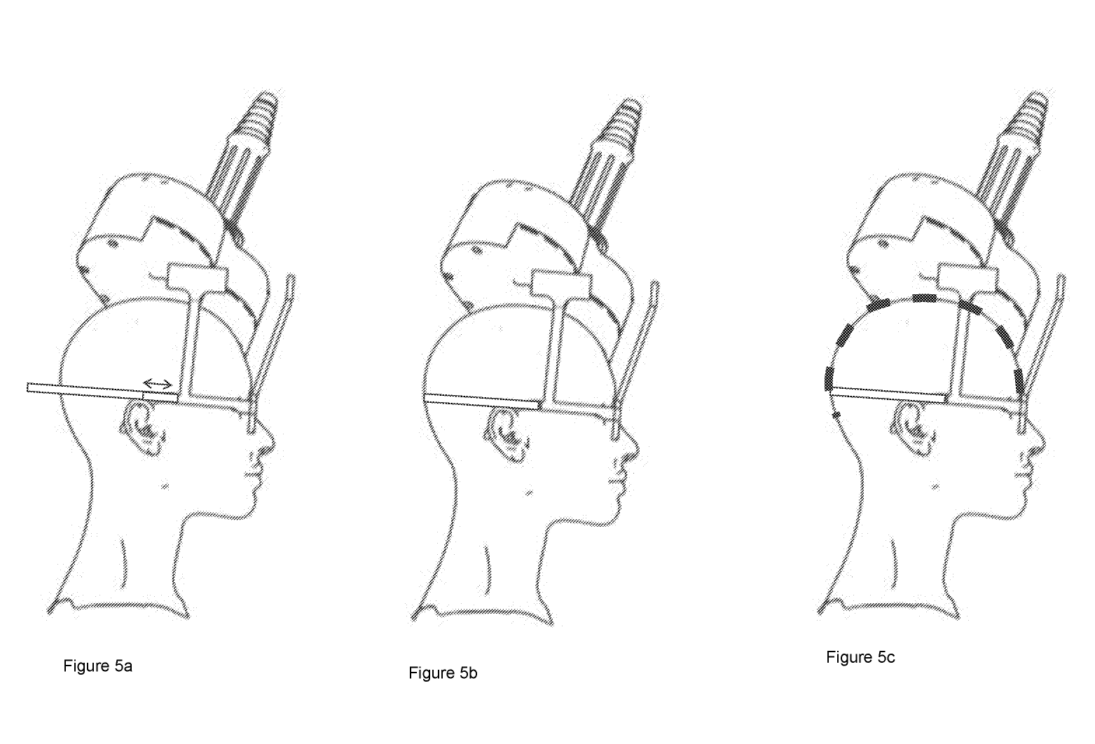

FIGS. 5A-C are schematic representations of illustrative embodiments of the present invention in use.

Referring to FIGS. 2A-2D the system comprises a wearable apparatus 12 arranged to be supported by a patient's nose and ears, memory and processor 14 and output arrangement 16. The memory and processor 14, forming components of a control arrangement, are preferably arranged in communication via a wireless or physical connection to the wearable apparatus 12.

The wearable apparatus 12 comprises a support arrangement 18 carrying a plurality of proximity sensors 20, preferably comprising time of flight sensors. Each of the proximity sensors preferably comprises an array of proximity sensors, where a greater number increases the positioning accuracy of the coil arrangement 10. The proximity sensors 20 are arranged such that the output from each of the sensors/sensor array converges meaning that at least two positional reference values are provided for the current or live position of the coil arrangement meaning that current positional information relating to the coil arrangement can be derived and compared to the stored preferred treatment position. The proximity sensors 20 are also supported at the periphery of a measurement zone and directed towards the measurement zone, which is beneficially above a patient's head.

The support arrangement is arranged to be supported by and seat on a wearer's nose and ears. The support structure 18 comprises a first seating portion 22 for seating onto a wearer's nose, and further comprises arms 24 extending to a distal end having a second seating portion 26 for seating onto a wearer's ear.

As can be seen in FIG. 5 the arms 24 may be adjustable in length. An extending arrangement is beneficially provided to enable adjustability in length. During initial set up of the system for use when determining the DCP which may be the treatment position, an input is made to the system identifying the relative adjustment in order that the correct position of the preaurical point is determined and thus finding the motor threshold is quicker and easier.

It can be seen that the support structure 18 is arranged to carry the plurality of proximity sensors 20 such that the proximity sensors emit an output into a zone around a patient's head, and into a zone in which the coil arrangement must be located. In order to achieve this the support structure 18 may comprise one or more proximity sensor carrying portions 28 arranged to extend away from a wearer's head such that the proximity sensors are spaced apart from a wearer's head and will not impede positioning of the coil. The proximity sensor carrying portions 28 therefore extend away from the arm 24, and are therefore spaced apart from the one or more arms. The sensor carrying portions may be in the form of a plate. Alternatively to the arrangement of first and second proximity sensor carrying portions being present as per the illustrative embodiment, it will also be appreciated that a single proximity sensor carrying portion may be provided having proximity sensors provided on a concave surface thereof. Beneficially there may be more than two sensor carrying portions.

The wearable apparatus 18 preferably comprises one or more markers 30 that provide reference points for a patient's head. This means that a control system can have repeatable and consistent information regarding one or more points relative to a patient's head irrespective of the exact position and orientation of a patient's head. A significant benefit in this regard is instead of a patient having markers physically marked on their head which is invasive and time consuming for each treatment, or alternatively wearing a cap comprising markers that is susceptible to moving or being positioned incorrectly or inconsistently, at least one point relative to the patient is always the same. The reference point that is preferably utilised is provided by the wearable device and is located adjacent the nasion which is at the upper end of a patient's nose. Therefore the support structure 18 is seated on the nasion meaning this reference point is immediately apparent to the control arrangement 14. Additional reference points are provided on the arms 24 representing the preaurical points, and adjustability of the arms means that an input can be made to the control system 14 to reflect the selected position of the arms and thus the preaurical points for that individual, where an algorithm in the control system determines the predicted motor threshold based on such inputs. This may take the form of a ruler on the adjustable section to determine the distance down from the seating portion 22 to the preauricular point. It will be appreciated that with respect to FIG. 5C the arms 24 may extend to the inion and/or a band 32 may extend over a wearer's head to provide a measurement from inion to nasion thereby providing the control system with additional information in order to provide a more accurate model of the head and thus enable determination of the treatment location to be made easier and quicker.

Accordingly, the wearable device preferably comprises multiple markers for use by the control system for more accurate modelling of a patient's head that may be utilised for increased accuracy in determination of the treatment location.

There are two distinct stages of operation associated with the present invention. A first stage is the set-up of the apparatus such that the coil arrangement is appropriately positioned for the requirement of an individual patient in order that the correct location in the brain is treated. The second stage is for subsequent patient treatment and the ability of the present invention to avoid the requirement for repeated determination of the treatment location. Instead, the second stage enables an operator to quickly position the coil arrangement in the correct location for effective treatment without the set up stage. Thus, for each patient, there is a single set-up stage which allows subsequent repeated treatments with ease.

In the first stage where the treatment location is determined for the first time, the following steps are preferably completed. The software of the control system has stored therein a representation of the support structure. The support structure is placed upon a wearer and the nasion of the wearer is known from the position of the marker 30 on the first seating portion 22 of the support structure 18. The arms are adjusted to sit on the wearer's ears, and the position measurement of the preauricular point from the markers 30 is fed into the control system 14. At this point a reference frame of the support structure 18 is provided thereby enabling the generation of a reference frame of the shape of the wearer's head. The accuracy may be increased by inputting information as presented with respect to FIGS. 5A-5C comprising a measurement of the inion relative to the preaurical points, and inion to nasion for example thereby enabling a better representation of the particular wearer's head to be generated.

The control system at this point can then calculate a predicted location for motor threshold determination. This is beneficial as an operator has a good indication of the location without being reliant upon experience. The position can be generated in various ways, for example by determining a plane between the nasion and preaurical points. From this a vector can be calculate extending perpendicular to the plane generated between the nasion and two preauricular points and extending from the midpoint between the two preauricular points in the direction of Z3 for example (shown in FIG. 1E). The origin of this vector is a reference point (RP) for the model of the patient's brain. The system based on the chosen head model next calculates the distance across the patient's head from the left to right pre-auricular points and calculates 20% of this distance. A new vector is projected from the RP to a point on the head model representing a movement across the circumference of the head from the position Z3 to the left preauricular point by the distance calculated in the previous step. The aim is then to get the center of this coil to position on this vector. This point is presented on the graphical display 16 meaning the operator is guided in movement of the coil arrangement 10 to this position.

Once the motor threshold is determined, then this location may be input as a value measured by the plurality of proximity sensors 20. An output may then be recorded in the control system 14 as represented in for example FIG. 3B where each proximity sensor comprises for example an array of twenty four individual proximity sensors.

The control system then guides an operator on the graphical display from the motor threshold point in a direction towards the nose by 5.5cm as per known and accepted determination of the treatment location (F3) as in FIG. 1E. This position is then recorded from the proximity sensors 20. FIG. 3B shows an example for each proximity sensor 20 which is made up of twenty four individual proximity sensors 20a in an array. Each individual proximity sensor 20a records time of flight information from the individual sensor to the coil arrangement. The control system takes a measurement of each coordinate, meaning that a graphical representation of the position of the coil arrangement 10 can be generated. Each of the proximity sensor arrays provides an output of distance to the coil arrangement, meaning a detailed live representation of the coil arrangement may be generated. The control system has stored therein information relating to the shape of the coil arrangement. An accurate 3D representation (sampling) of the relative position of the coil arrangement 10 relative to the proximity sensors can therefore be generated by a matching algorithm utilising the outputs of the proximity sensors and the known shape of the coil arrangement. This means that for each individual patient accurate positioning of the coil arrangement can be achieved relative to the proximity sensors irrespective of the position of the head, meaning an accurate DCP location can be achieved for each session, and preferably each treatment session.

As presented in FIGS. 4A and 4B, the proximity sensor 20 in the form of an array of twenty four sensors is represented in top view and side view respectively. In addition the outline of the coil arrangement 10 is also presented. Accordingly, each individual proximity sensor 20a in the array measures time of flight information to the coil arrangement thereby building up a picture of the relative position and orientation of the coil arrangement 10 relative to the coordinate system of the patient.

It will be appreciated and understood that the position of the motor threshold may be determined through other means, such as for example physical measurement rather than through use of the capability of being guided using the proximity sensors. In addition, other locations on a body may be treated using Magnetic Stimulation and other different techniques may be utilised for determining the respective treatment location.

Once the system has a recorded set of coordinates from the proximity sensors for a particular individual patient, subsequent treatment is simplified. The support arrangement 18 is positioned onto the patient, and the required information regarding the adjustment of the arms 24 is made. The control system 14 has stored therein the coordinates for the treatment position, and as the proximity sensors 20 are always in the same position relative to the head then a representation of the treatment position of the coil arrangement can be presented on the display 16 irrespective of the precise positioning of the patient's head. As the coil arrangement is introduced into the zone of the proximity sensors, continuous measurements can be made and compared to the stored settings for the treatment position. An operator can therefore be guided to the treatment location, which may be achieved through graphical representation on the display 16. Once the coordinates stored in the memory match those measured at that moment in time, an indication such as an audible output may be provided to the operator and the coil arrangement 10 locked into position. Treatment can then be performed.

Aspects of the present invention have been described by way of example only and it will be appreciated that modifications and variations may be made by the skilled addressee without departing from the scope of protection afforded by the appended claims.

1. A system for enabling positioning of a Magnetic Stimulation (MS) coil arrangement relative to a wearer's body to a Desired Coil Position (DCP) comprising:
a wearable apparatus comprising a support structure for positioning onto the body of a wearer, the support structure carrying a plurality of proximity sensors for measuring the proximity of a MS coil arrangement relative thereto to provide measured proximity values;
a processor configured to compare proximity values for each of the plurality of proximity sensors representing the proximity of a MS coil arrangement to the sensors in the DCP to the measured proximity values and determine when the measured proximity values match the DCP proximity values thereby indicating a MS coil arrangement is in the DCP;
an output arrangement for providing an indication to an operator when the MS coil arrangement is in the DCP.

2. A system according to claim 1 wherein the output arrangement comprises a graphical display and presents on the graphical display a graphical representation of a MS coil arrangement in the DCP and a graphical representation of the current MS coil arrangement position.

3. A system according to claim 2 further arranged to present a graphical representation of the wearer's body on the graphical display.

4. A wearable apparatus for use in positioning of a Magnetic Stimulation (MS) coil arrangement relative to a wearer's body, the wearable apparatus comprising a support structure for positioning onto the body of a wearer, the support structure carrying a two or more proximity sensors for measuring the proximity of a MS coil arrangement.

5. A wearable apparatus according to claim 4 wherein the support structure is arranged to support the two or more proximity sensors at the periphery of a measurement zone, and the two or more sensors are supported such that they are inwardly directed into the measurement zone.

6. A wearable apparatus according to claim 4 wherein the support structure comprises a first seating portion for seating onto a wearer's nose, and further comprises at least one arm extending to a distal end having a second seating portion for seating onto a wearer's ear.

7. A wearable apparatus according to claim 6 wherein the arm is adjustable in length.

8. A wearable apparatus according to claim 4 wherein the support structure is arranged such that when positioned on the body the two or more proximity sensors are spaced apart from the body.

9. A wearable apparatus according to claim 4 wherein the support structure comprises one or more sensor carrying portions arranged to extend away from a wearer's body such that the proximity sensors are spaced apart from a wearer's head.

10. A wearable apparatus according to claim 9 wherein the sensor carrying portion extends away from the arm.

11. A wearable apparatus according to claim 6 wherein the proximity sensors are spaced apart from the one or more arms.

12. A wearable apparatus according to claim 4 wherein one or each of the plurality of proximity sensors are provided as one or more sensor arrays.

13. A wearable apparatus according to claim 4 comprising one or more markers that provide reference points for a wearer's body.

14. A wearable apparatus according to claim 13 comprising multiple markers.

15. A wearable apparatus according to claim 14 wherein a marker is provided on the or each of the arms of the wearable device.

16. A wearable apparatus according to claim 4 wherein the proximity sensors comprise time of flight sensors.

17. A method of positioning a Magnetic Stimulation (MS) coil arrangement relative to a wearer's body to a Desired Coil Position (DCP) comprising:
positioning a wearable apparatus comprising a support structure onto the body of a wearer, the support structure carrying a plurality of proximity sensors for measuring the proximity of a MS coil arrangement relative thereto to provide measured proximity values;
comparing the measured proximity values to each of reference values representing the proximity of a MS coil arrangement to the sensors in the DCP;
outputting an indication to an operator when the measured proximity values match the DCP proximity values thereby indicating a MS coil arrangement is in the DCP.
